(19) United States
(12) Patent Application Publication
(10) Pub. No.: US 2025/0087325 A1
(43) Pub. Date: Mar. 13, 2025

(54) METHOD, APPARATUS, DEVICE AND STORAGE MEDIUM OF INFORMATION INTERACTION

(71) Applicant: Beijing Zitiao Network Technology Co., Ltd., Beijing (CN)

(72) Inventors: Wei TU, Beijing (CN); Yihang TANG, Beijing (CN)

(21) Appl. No.: 18/882,670

(22) Filed: Sep. 11, 2024

(30) Foreign Application Priority Data
Sep. 11, 2023 (CN) 202311170328.3

(51) Int. Cl. G16H 15/00; G16H 40/67; G16H 50/20; G16H 50/30

(52) U.S. Cl. CPC G16H 15/00; G16H 40/67; G16H 50/20; G16H 50/30

(57) ABSTRACT

The embodiment of the invention provides an information interaction method and device, equipment and a storage medium. The method includes the following steps: in response to a consultation initiating operation, providing an interactive interface of a user and a digital assistant to be used for health consultation of the user; after the user interacts with the multi-round of the digital assistant, determining whether a presentation condition of triggering the health consultation small junction is met or not; if it is determined that the presentation condition of triggering the health consultation small junction is met, providing a health consultation small junction in the interaction interface, wherein the health consultation small junction has a structured format and is generated based on at least interactive information of the user and the digital assistant.

### CROSS REFERENCE

This application claims priority of the Chinese patent application No. 202311170328.3, entitled “METHOD, APPARATUS, DEVICE OF INFORMATION INTERACTION AND STORAGE MEDIUM” filed on Sep. 11, 2023, the entire content of which is incorporated herein by reference.

### FIELD

Example embodiments of the present disclosure generally relate to the field of computers, and in particular, to methods, apparatuses, devices and computer-readable storage media of information interaction.

### BACKGROUND

With the development of Internet technologies, it is also expected to enable people to enjoy remote and online services in terms of health consultation. Online medical care improves the supply of high-quality medical resources with high efficiency and low cost, helps medical institutions to expand the radiation range of the medical services, and enables user to obtain the medical guidance of the authoritative professional at any time. Currently, some websites, terminal applications, and the like, attempt to provide online health consultation services. However, the interactivity and expertise in the online health consultation still need to be improved.

### SUMMARY

In a first aspect of the present disclosure, a method of information interaction is provided. The method comprises: in response to a consultation initiation operation, providing an interaction interface between a user and a digital assistant for health consultation of the user; determining, after a multi-round interaction between the user and the digital assistant, whether a presentation condition triggering a health consultation summary is satisfied; providing the health consultation summary in the interaction interface, in response to a determination that the presentation condition triggering the health consultation summary has been satisfied, the health consultation summary having a structured format and generated based at least on interaction information between the user and the digital assistant.

In a second aspect of the present disclosure, an apparatus for information interaction is provided. The apparatus comprises: an interface provision module, configured to, in response to a consultation initiation operation, provide an interaction interface between a user and a digital assistant for health consultation of the user; a presentation determining module, configured to determine, after a multi-round interaction between the user and the digital assistant, whether a presentation condition triggering a health consultation summary is satisfied; and a summary presentation module, configured to provide the health consultation summary in the interaction interface, in response to a determination that the presentation condition triggering the health consultation summary has been satisfied, the health consultation summary having a structured format and being generated based at least on interaction information between the user and the digital assistant.

In a third aspect of the present disclosure, an electronic device is provided. The device includes at least one processing unit; and at least one memory coupled to the at least one processing unit and storing instructions for execution by the at least one processing unit. The instructions, when executed by the at least one processing unit, cause the device to perform the method of the first aspect.

In a fourth aspect of the present disclosure, a computer-readable storage medium is provided. The computer-readable storage medium stores a computer program, and the computer program is executable by the processor to implement the method of the first aspect.

It should be understood that the content described in this content section is not intended to limit the key features or important features of the embodiments of the present disclosure, nor is it intended to limit the scope of the present disclosure. Other features of the present disclosure will become readily understood from the following description.

### BRIEF DESCRIPTION OF DRAWINGS

The above and other features, advantages, and aspects of various embodiments of the present disclosure will become more apparent from the following detailed description taken in connection with the accompanying drawings. In the drawings, the same or similar reference numbers refer to the same or similar elements, wherein:

FIG. 1 illustrates a schematic diagram of an example environment in which embodiments of the present disclosure can be implemented;

FIG. 2A to FIG. 2E are schematic diagrams of examples of an information interaction interface according to some embodiments of the present disclosure;

FIG. 3 illustrates a flowchart of a process of generating a health consultation summary according to some embodiments of the present disclosure;

FIG. 4 illustrates a schematic diagram of a process of a dialog between a digital assistant and a user according to some embodiments of the present disclosure;

FIG. 5 illustrates a schematic diagram of an architecture for providing a health consultation summary according to some embodiments of the present disclosure;

FIG. 6 illustrates a flowchart of a process for information interaction according to some other embodiments of the present disclosure;

FIG. 7 illustrates a block diagram of an apparatus for information interaction according to some embodiments of the present disclosure; and

FIG. 8 illustrates a block diagram of an electronic device in which one or more embodiments of the present disclosure may be implemented.

### DETAILED DESCRIPTION

Embodiments of the present disclosure will be described in more detail below with reference to the accompanying drawings. While certain embodiments of the present disclosure are shown in the accompanying drawings, it should be understood that the present disclosure may be implemented in various forms, and should not be construed as limited to the embodiments set forth herein, but rather, these embodiments are provided for a more thorough and complete understanding of the present disclosure. It should be understood that the drawings and embodiments of the present disclosure are for illustrative purposes only and are not intended to limit the scope of the present disclosure.

In the description of the embodiments of the present disclosure, the terms “including” and the like should be understood to include “including but not limited to”. The term “based on” should be understood as “based at least in part on”. The terms “one embodiment” or “the embodiment” should be understood as “at least one embodiment”. The term “some embodiments” should be understood as “at least some embodiments”. Other explicit and implicit definition may also be included below.

Herein, unless explicitly stated, “in response to A” performing a step does not mean that this step is performed immediately after “A”, but may include one or more intermediate steps.

It may be understood that the data involved in the technical solution (including but not limited to the data itself, the obtaining, using, storing or deleting of the data) should follow the requirements of the corresponding laws and regulations and related regulations.

It can be understood that before using the technical solutions disclosed in the embodiments of the present disclosure, relevant user should be informed of the types, use ranges, usage scenarios, and the like of the information related to the present disclosure in an appropriate manner according to relevant laws and regulations, and the authorization of the related user may be obtained, wherein the relevant users may include any type of rights body, such as individuals, businesses, and groups.

For example, when an active request of a user is received, prompt information is sent to the related user to explicitly prompt the related user, and the operation requested to be performed will need to obtain and use the information of the related user, so that the related user can autonomously select whether to provide information to software or hardware such as electronic device, application, server or storage medium and etc., executing the operation of the technical solution of the present disclosure according to the prompt information.

As an optional but non-limiting implementation, in response to receiving an active request of a related user, a manner of sending prompt information to the related user may be, for example, a pop-up window, and prompt information may be presented in a text manner in the pop-up window. In addition, the pop-up window may further carry a selection control for the user to select “agree” or “not agree” to provide information to the electronic device.

It may be understood that the foregoing notification and obtaining a user authorization process are merely illustrative, and do not constitute a limitation on implementations of the present disclosure, and other manners of meeting related laws and regulations may also be applied to implementations of the present disclosure.

As used herein, the term “model” may learn an association relationship between respective inputs and outputs from training data such that a corresponding output may be generated for a given input after training is complete. The generation of the model may be based on machine learning techniques. Deep learning is a machine learning algorithm that processes inputs and provides corresponding outputs by using a multi-layer processing unit. The neural network model is one example of a deep learning-based model. As used herein, a “model” may also be referred to as a “machine learning model,” a “learning model,” a “machine learning network,” or a “learning network,” these terms are used interchangeably herein.

During the online health consulting, users often go through multiple sessions in order to provide more information for assisting in judgment, or different aspects of a question. However, most of the current model-based digital assistants do not support information organization capability, that is, the digital assistant can only answer based on the user's questions, and lack the ability to organize these questions and the contents of the question and answer. For example, a consultation result can only be provided to the user by, for example, listing a historical session record. In this way, in the scenario of allowing the user to perform the multi-round questioning, it is difficult for the user to intuitively and conveniently read the consulted question, and obtain a response result. For example, the user needs to understand the historical question and the corresponding question and answer, then the relevant information needs to be obtained by, for example, continuously scrolling the session interface.

According to an embodiment of the present disclosure, an improved solution for information interaction is provided. In the solution, in response to the consultation initiation operation, an interaction interface between the user and the digital assistant is provided for a health consultation of the user; after a multi-round interaction between the user and the digital assistant, whether a presentation condition triggering a health consultation summary is satisfied is determined; the health consultation summary is provided in the interaction interface, in response to a determination that the presentation condition triggering the health consultation summary has been satisfied, the health consultation summary having a structured format and generated based at least on interaction information between the user and the digital assistant. Therefore, after the condition of the interaction situation is satisfied, the consultation content and the consultation result can be intuitively and clearly provided for the user by using the health consultation summary with the structured format, so that they can be read conveniently by the user, and improve the information acquisition efficiency of the user.

Some example embodiments of the present disclosure will be described in detail below with reference to the accompanying drawings.

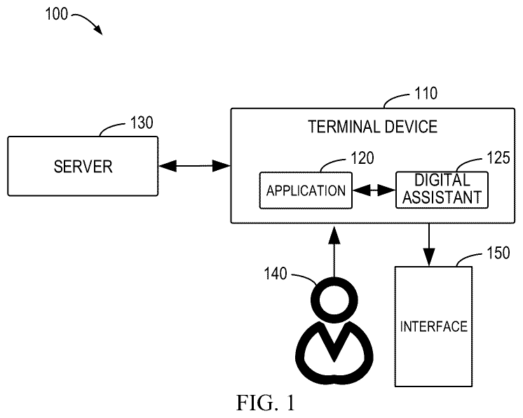

FIG. 1 illustrates a schematic diagram of an example environment 100 in which embodiments of the present disclosure can be implemented. In this example environment 100, an application 120 is installed in the terminal device 110. The user 140 may interact with the application 120 via the terminal device 110 and/or an attached device of the terminal device 110.

In some embodiments, the application 120 may be downloaded, installed on the terminal device 110. In some embodiments, the application 120 may also be accessed in other manners, such as web page access. In the environment 100 of FIG. 1, if the application 120 is launched, the terminal device 110 may present the interface 150 of the application 120. The interface 150 may include various user interfaces that may be provided by the application 120. Interface 150 is also sometimes referred to as a client interface.

In embodiments of the present disclosure, the application 120 includes any suitable application capable of providing a health consultation service. In some embodiments, the application 120 may implement a dialog interaction with the user 140 based on the digital assistant 125 to provide a health consultation service to the user 140. In some embodiments, the application 120 may provide other services in addition to the interaction of the digital assistant 125, which is not limited in this respect.

In some embodiments, terminal device 110 may communicate with server 130 to enable provision of services to applications 120 and/or digital assistant 125. The terminal device 110 may be any type of mobile terminal, fixed terminal, or portable terminal, including a mobile phone, a desktop computer, a laptop computer, a notebook computer, a netbook computer, a tablet computer, a media computer, a multimedia tablet, a personal communication system (PCS) device, a personal navigation device, a personal digital assistant (PDA), an audio/video player, a digital camera/camcorder, a television receiver, a radio broadcast receiver, an electronic book device, a gaming device, or any combination of the foregoing, including accessories and peripherals of these devices, or any combination thereof. In some embodiments, the terminal device 110 can also support any type of interface for a user (such as a “wearable” circuit, etc.). The application 130 may be various types of computing systems/servers capable of providing computing power, including, but not limited to, mainframes, edge computing nodes, computing devices in a cloud environment, and so forth.

It should be understood that the structures and functions of the various elements in the environment 100 are described for illustrative purposes only and do not imply any limitation to the scope of the present disclosure.

In an embodiment of the present disclosure, the digital assistant 125 is configured to interact with a user. An interaction interface, also referred to as an interaction window or a dialog window, between the user and the digital assistant 125 is presented in the client interface. The digital assistant 125, as an intelligent assistant, has an intelligent dialog and information processing capability. In the interaction interface, the user inputs an interaction message, and the digital assistant 125 provides a response message in response to user input. In application 120, the digital assistant 125 may be launched or awakened in an appropriate manner (e.g., a shortcut key, button, or voice) to present an interaction interface with the user. The interaction interface may include an interface element for information interaction, such as an input box, a message list, a message bubble, and the like.

In embodiments of the present disclosure, digital assistant 125 is used to provide services related to health consultation, such a digital assistant 125 may sometimes be referred to as a “digital doctor”. The user may send a health consultation requirement to the digital assistant 125 by using a simple text or voice input, and may obtain a real-time response of the digital assistant 125 to perform medical knowledge questioning and answering, health consultation, or general health management. In this way, the digital assistant 125 can be used to achieve a high efficiency and a low cost to improve the supply of high-quality medical resources, help the medical institution to expand the radiation range of the medical service, and enable the user to obtain the guidance of the authoritative professional at any time.

In some embodiments, the digital assistant 125 may determine a response to the user's question by means of a model. In some embodiments, the model may be trained based on a general corpus and a specialized corpus for the medical field, enabling reachable, credible, understandable medical knowledge publicity and health consultation guidance. The specialized corpus in the medical field may include, for example, national and international authoritative clinical guidelines, documents, textbooks, clinical doctor-patient communication question-answer data, and the like. In some embodiments, the training of the medical safety related mechanism may be strengthened during model training, so that the model can have the capability of providing credible health consultation suggestions, and a comprehensive safety test is passed. In some embodiments, when evaluating the performance of the digital assistant 125, the capability of the digital assistant 125 in terms of communication expression ability and humanistic care and friendliness may also be evaluated, so that the digital assistant 125 can realize the aspects of empathy with a user, understanding of user requirements, detailed and patient communication and the like, which is advantageous for alleviating the problem of short communication time and insufficient communication.

Some example embodiments of the present disclosure will be described below with continued reference to the accompanying drawings.

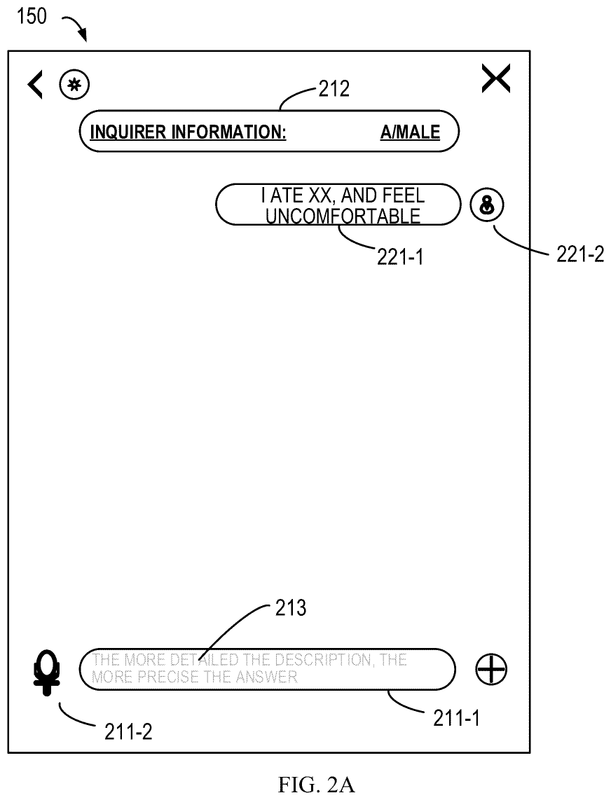

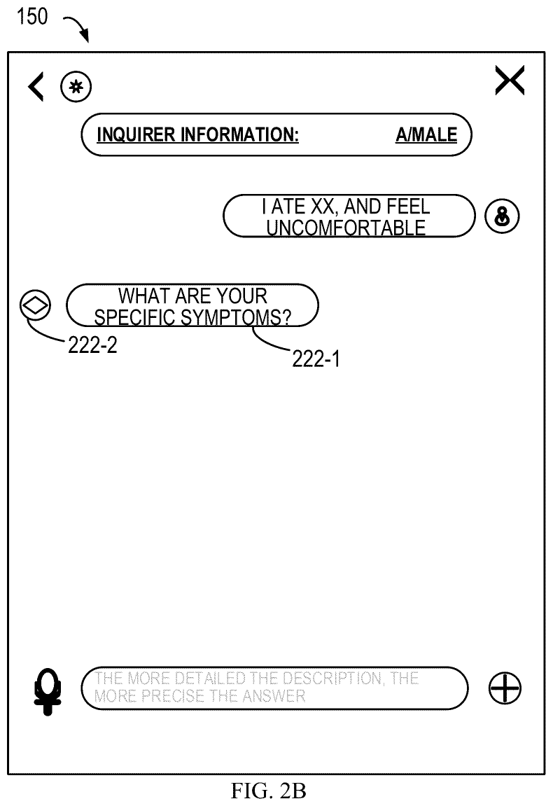

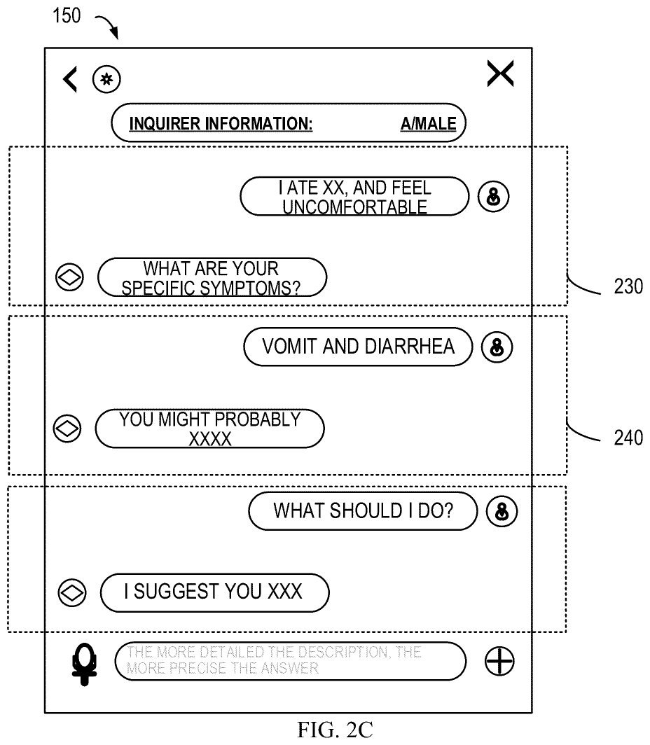

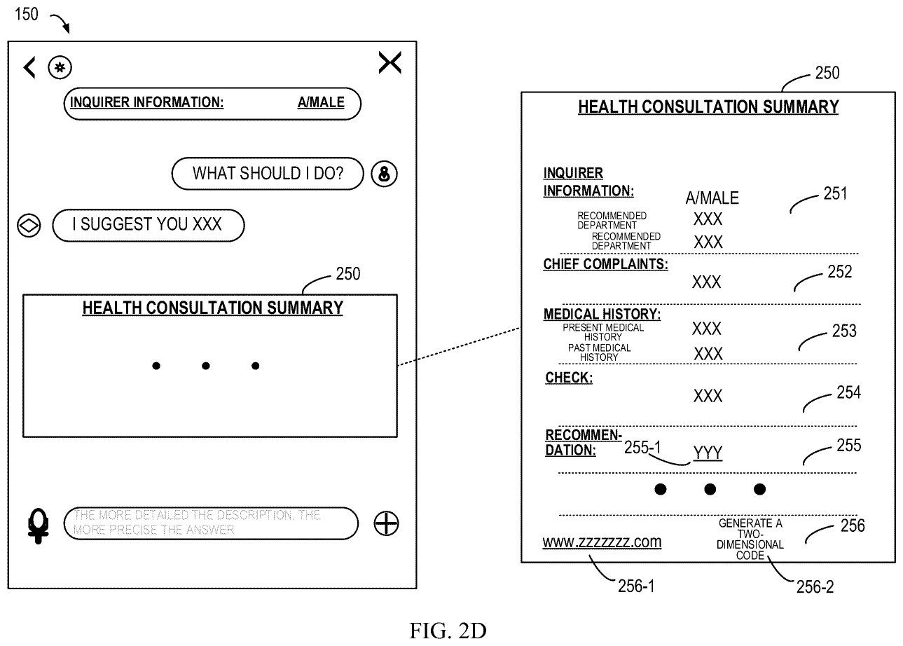

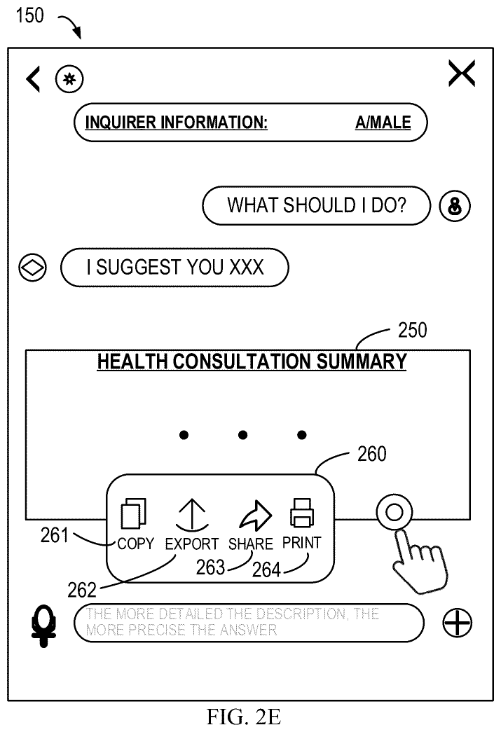

In the following, for a better understanding of the embodiments of the present disclosure, reference will be made to the examples of the information interaction interface shown in FIG. 2A to FIG. 2E for description. It should be understood that the information interaction interface in these figures is merely an example, and various interface designs may actually exist. Individual graphical elements in the interface may have different arrangements and different visual representations, one or more of which may be omitted or replaced, and one or more other elements may also be present. Embodiments of the present disclosure are not limited in this respect.

As noted above, the application 120 may be used by the user 140. In an embodiment of the present disclosure, in response to the consultation initiation operation, the consultation initiation operation may be, for example, the user clicks the application 120 (or, enter the specific ‘consultation function’ in the application 120), the terminal device 110 may provide an interaction interface (for example, the interface 150) with the digital assistant 125 for the user 140 for the health consultation of the user 140.

The user 140 may perform a session with the digital assistant 125 through the interface 150 to, for example, initiate a consultation operation. For example, the user 140 may implement, for example, the purpose of health consultation by sending the text information to the digital assistant 125 after inputting the text information in the interface 150.

For example, refer to FIG. 2A. The interface 150 shown in FIG. 2A may be provided to the user 140 after the user 140 performs a consultation initiation operation. The input box 211-1 for sending text information and the control 211-2 for voice information may be included in the interface 150. For example, user 140 may send “consultation content” in the form of text by inputting information in the input box 211-1. Similarly, after activating the control 211-2, the user 140 may input “consultation content” through the manner of voice information.

In some embodiments, in order to better guide the user 140, the guidance prompt information 213 may also be revealed in the input box 211-1 to assist the user in better inputting the “consultation content”.

Accordingly, after sending the consultation content, the terminal device 110 may present the consultation content in the interface 150. In some embodiments, when the terminal device 110 presents the consultation content, the presentation may be implemented by using a combination of the actual content and the identification information of the user 140 (for example, avatar, nickname information, etc.). In this way, the sender of the “consultation” content can be presented more clearly with the actual content. For example, in the interface 150 shown in FIG. 2A, after the user 140 sends the consultation content “I ate XX, and fell uncomfortable” through the input box 211-1, the session message 221-1 and the presentation style, such as the identifier 221-2 of the avatar of the user 140 may be presented in the interface 150. Thus, the user 140 may consult with the digital assistant 125 to obtain desired content (e.g., knowledges, solutions, etc.).

In some embodiments, the terminal device 110 may further reveal the information card 212 in the interface 150 to present relevant information of the user 140 with the information card 212.

Further, after receiving the consultation content of the user 140 (for example, the session message 221-1), the digital assistant 125 may reply to the consultation content. Typically, the reply may be a response that is made directly for the content that the user 140 consults, or may be “subsequent questioning” information for obtaining more information in order to answer the question inquired by the user 140. For example, if the consultation content provided by the user 140 is clear enough and definite enough, so that the digital assistant 125 may directly reply based on the information currently provided by the user 140, the digital assistant 125 may directly generate and present the answer in the interface 150 to response to the user 140. In another example, if the digital assistant 125 determines that the currently obtained information is insufficient to answer the user 140, the digital assistant 125 may select a manner of providing subsequent questioning information, and require the user 140 to provide more information for reference.

In some embodiments, the digital assistant 125 generates a response to the user 140 based on the model. For example, the digital assistant 125 may obtain the capability of providing a response to the user 140 based on the model. In some embodiments, the digital assistant 125 may obtain the capability of providing a response based on a language model (LM) or based on other suitable model (e.g., parse semantics, the capability of querying related knowledge and content based on the semantic parsing result to provide a response). The digital assistant 125 may generate a prompt input (Prompt) based on the inquired question input by the user 140. Then, the digital assistant 125 may uses the LM to process the prompt input to generate a processing result corresponding to the prompt input. Further, the digital assistant 125 may present the processing result to the user 140 to enable the interaction with the user 140.

The model may be deployed on the terminal device 110 or may be accessed by the terminal device 110. Alternatively, the terminal device 110 may communicate with the model to provide the application management service for the user 140 using the model. For example, the terminal device 110 may send a prompt input to the model based on the interaction information between the user 140 and the digital assistant 125, and obtain a processing result of the model. Alternatively, or additionally, the digital assistant 125 itself may also be obtained based on a model. The digital assistant 125 may process the session message input by the user 140 to obtain a requirement of the user 140, for example, a problem expected to be consulted by the user 140, and the like. The model may be configured locally or independently of the terminal device 110 according to requirements, and the present disclosure is not intended to be limited thereto.

For example, refer to FIG. 2B. After the user 140 sends the session message 221-1 at the interface 150, for example, the digital assistant 125 determines that it is currently unable to reply based on the session message 221-1. The digital assistant 125 may send session information 222-1 in the interface 150 for “subsequent questioning”. For example, the user 140 is subsequently inquired about specific “symptoms”. Similarly, the terminal device 110 may also present a response content made by digital assistant 125 by using a combination of identification and session information. For example, in interface 150, the response made for digital assistant 125 is presented in the form of a combination of session message 222-1 and identifier 222-2. In this way, the user 140 may distinguish the subject who sent the session message by the identifier associated with the session message. In some embodiments, the terminal device 110 may further adjust the background color of the session message (for example, the background colors of the session message sent by the user 140 and the terminal device 110 are different) and the presentation position (for example, in the interface 150, the session message sent by the user 140 is displayed on the right side of the interface, and the session message sent by the terminal device 110 is displayed on the left side of the interface) of the session message, to facilitate the user 140 to distinguish the session message.

In an embodiment of the present disclosure, the user 140 initiates a question and the digital assistant 125 responds to a question, which may be regarded as a session in which the user 140 completes one round with the digital assistant 125, or one round of interaction. During the interaction process, the digital assistant 125 may obtain more information from the user 140 through a multi-round dialog, or answer different aspects of the consultation content for the user 140. For example, after completing the session of one round, the user 140 may start the next round of session by continuing to question or answer. In some embodiments, the interface 150 may continuously control the area in the interface 150 to scroll down based on the performing of the session between the user 140 with the digital assistant 125 to successively present the specific session content in each round. Further, the terminal device 110 may detect an interaction situation between the digital assistant 125 and the user 140, for example, detect an interaction round between the digital assistant 125 and the user 140.

For example, refer to FIG. 2C. In FIG. 2C, the terminal device 110 may detect an interaction round between the digital assistant 125 and the user 140. For example, the terminal device 110 detects that the digital assistant 125 and the user 140 complete three rounds of interaction, i.e., the dialog round 230, the dialog round 240, and the dialog round 250.

Further, the terminal device 110 may determine, based on a current interaction situation (for example, a number of interaction rounds, whether it is enough to complete the consultation, or the like), whether a presentation condition triggering the health consultation summary is satisfied. If satisfied, the terminal device 110 may provide a health consultation summary in the interface 150. Therefore, the proper timing for giving the conclusion can be determined by judging the completeness and the depth of the dialog information, and the user information is prevented from being excessively collected, and the user experience is improved.

For example, refer to FIG. 2D. If satisfied, the terminal device 110 may provide a health consultation summary 250 in the interface 150.

In some embodiments, the terminal device 110 may determine whether to present the health consultation summary 250 based on whether a keyword for triggering the health consultation summary can be detected from the interaction information. The keywords may be, for example, keywords sent by the user 140, such as “end the consultation”, “generate a summary”, etc., indicating the completion of a consultation, or requiring the generation of a summary. In a scenario that the terminal device 110 determines that the interaction information includes these keywords, it is determined that the presentation condition triggering the health consultation summary 250 has been satisfied (for example, a form of sending a session message by using the digital assistant 125), and the health consultation summary 250 is presented for the user 140.

In some embodiments, the terminal device 110 may also determine, based on the interaction information, whether the health consultation can obtain a predicted consultation conclusion, to determine whether to present the health consultation summary 250. For example, the terminal device 110 may determine whether the digital assistant 125 may generate a predicted consultation conclusion (e.g., a health recommendation that satisfies the credibility requirement can be provided) based on information currently provided by the user 140. If yes, the terminal device 110 may determine that a condition of presenting the health consultation summary is satisfied to determine that the health consultation summary 250 is presented for the user 140. In some embodiments, the terminal device 110 may further determine whether the trigger condition is satisfied by estimating whether the probability of generating a predicted consultation conclusion reaches the probability threshold. For example, based on the currently obtained interaction information, the problem that the user 140 consults may be predicted that the probability of a certain consultation conclusion exceeds a previous probability threshold, then it is determined that the triggering condition is satisfied.

The terminal device 110 may also provide the health consultation summary 250 in a scenario where the user 140 requires or the current consultation has met the requirement of the user 140. Thus, it is convenient for the user 140 to intuitively and conveniently understand the consultation process and the consultation result by using the health consultation summary 250.

In some embodiments, in a scenario where the terminal device 110 provides the health consultation summary 250 based on the foregoing manner (for example, judging whether the presentation condition triggering the health consultation summary has been satisfied), the terminal device 110 may further present, before providing the health consultation summary 250, a confirmation request for the end of the health consultation in the interface 150. If the user 140 confirms for the confirmation request, the digital assistant 125 may then respond to this and provide a health consultation summary 250. Thus, the user may interact with the user before providing the health consultation summary 250 to determine whether the user 140 truly and actually completed the consultation, or the user 140 expects to end the consultation. Therefore, in order to meet the actual requirements of the user 140, mistakenly interrupting and ending the health consultation (or mistakenly providing the health consultation summary 250) are avoided, and the user experience of the user 140 is improved.

In the embodiments of the present disclosure, the health consultation summary 250 has a structured format and is generated based at least on interaction information between user 140 and the digital assistant 125. In some embodiments, the terminal device 110 may select at least one field from the plurality of predetermined fields based on the interaction information between the user 140 and the digital assistant 125, where each field indicates a type of information to be filled in. For example, the terminal device 110 may determine the plurality of fields based on a consultation answer made by the digital assistant 125 for the user 140, for example, a department recommended for the user 140, a check recommended to the user 140, and specific information provided by the user 140. For example, in a scenario where the digital assistant 125 provides a recommended department, the field may include a “recommended department”. Accordingly, the terminal device 110 may extract the key information corresponding to the field from the interaction information (for example, the dialog between the user 140 and the digital assistant 125) based on the fields, to generate the health consultation summary 250.

In some embodiments, the terminal device 110 may extract the key information from at least one of the following: a textual dialog between the user 140 and the digital assistant 125, or non-text information input by the user 140 (for example, voice information and image information). For example, in a scenario where the user 140 describes a symptom he or she has in a conversation, the field may be a “primary complaint”. In some embodiments, the fields may relate to primary complaints, medical history, check items, and the like. For example, the field may be a check of type A, such that the user 140 may understand, based on the field in the health consultation summary 250, whether a check of type A has been made, as well as a result of the check of type A. For example, the user 140 may also provide the consultation content by directly uploading a picture of the medical record and a picture of a question. Accordingly, the terminal device 110 may also extract key information related to the user 140 based on these contents. Therefore, the terminal device 110 may summarize the key information and determine a field of the content presented in the health consultation summary 250 based on the current consultation situation or the request of the user 140.

In some embodiments, the terminal device 110 may further determine an actual used field through some pre-maintained policies, to improve the use value of the field and improve more information. For example, based on the predicted disease, the content that the user 140 may expect to focus on, learn about is determined based on historical habits of the user 140, reference information provided by the user 140, and the like. Accordingly, the terminal device 110 may set a field corresponding to these contents.

In some embodiments, the terminal device 110 may determine, based on the interaction information between the user 140 and the digital assistant 125, a disease prediction corresponding to the health consultation of the user. For example, the terminal device 110 may determine the relevant field based on a conclusion made by the digital assistant 125 for the consultation of the user 140 (e.g., you may be “stomach bloating”). For example, fields related to “stomach bloating” such as check item, age, past medical history, etc. Therefore, the fields included in the health consultation summary 250 are determined in association based on the disease prediction result, so as to avoid the use of irrelevant fields and improve the use value of the fields. In some embodiments, a field selection module may be pre-trained, so that the terminal device 110 may select a field by using the field selection module. The field selection module may be pre-trained based on real cases such as medical cases, check reports and the like, so that the field selection module may dynamically select an appropriate field according to the interaction information and field requirements in the real check report, so that the generated report is more practical and professional.

For example, refer to FIG. 2D. The health consultation summary 250 includes a plurality of display areas (for example, display areas 251 to 255), and each display area may correspond to display content under a field item (or a set of fields having an association relationship). For example, in the area 251, content related to the field “consultant information” may be displayed. In the area 252, content related to the field “primary complaint” may be displayed. In the area 253, content related to the field “medical history” may be displayed. In the area 254, content related the field “Check” may be displayed. In the area 255, content related to the field “recommendation” may be displayed.

Further, the terminal device 110 may extract the actual content corresponding to the field from, for example, the interaction information based on the determined field, or may be presented in the specific content under the field item in the health consultation summary 250. It should be understood that based on different arrangement logic, one field may also be content to which another field belongs. For example, in a scenario where the field is primary complaint, medical history, and check information, the content corresponding to the field may be all content corresponding to the primary complaint field, the medical history field, and the check information field.

For example, a combination of fields and corresponding content may be:

### Medical History and Check Information

Medical history: present medical history, past medical history, personal history, and the like.
Check information: physical examination, specialized check, laboratory tests and imaging examination, and the like.

Generally, the terminal device 110 may arrange presentation positions of respective fields based on the number of fields included, and the content length of the content corresponding to each field, to generate the health consultation summary 250 having the structured format.

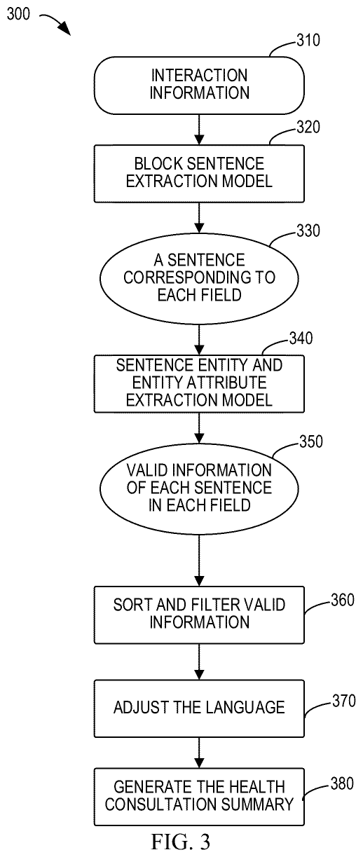

For this, refer to FIG. 3. FIG. 3 illustrates a flowchart of a process 300 of generating a health consultation summary according to some embodiments of the present disclosure.

At block 320, the terminal device 110 may extract the sentence 330 corresponding to each field from the interaction information between the user 140 and the digital assistant 125 by using a block sentence extraction model. The interaction information may be split into a plurality of sentences based on punctuation marks (e.g., commas, periods) in the interaction information. Then, the terminal device 110 performs block annotation on each sentence in the split document by using a block sentence extraction model, and the block may include, for example, a primary complaint, a present medical history, a past medical history, a physical examination, a specialized check, and the like.

At block 340, the terminal device 110 may extract the valid information 350 of each sentence in each field from the sentence 330 corresponding to each field based on the sentence entity and the entity attribute extraction model. For example, the terminal device 110 uses the entity extraction model to extract valid information such as an entity and an entity attribute of the sentence segmented into blocks.

At block 360, the terminal device 110 may, for each field, exclude and filter the valid information 350 of each sentence in each extracted field, so that the valid information is logical and readable.

In block 370, the terminal device 110 may adjust the text expression for the sorted and filtered valid information, for example, adjust the content of the valid information to be closer to the representation of the natural language form. Thus, the readability of the content is further improved. Therefore, through the semantic understanding and information extraction technology of the deep learning, the unstructured health consultation dialog between the user 140 and the digital assistant 125 is converted into a structured health consultation summary 250 which is easy to understand and standard and has practical value.

At block 380, the terminal device 110 associates these contents with a field, determines a field, and a presentation position of the content corresponding to the field to ultimately form a health consultation summary 250.

In some embodiments, the terminal device 110 may utilize a summary plugin to generate a health consultation summary 250. The summary plug-in is configured to generate the health consultation summary 250 based on the interaction information between the user 140 and the digital assistant 125 (e.g., the consultation content input by the user 140, and the response content returned by the digital assistant 125). The terminal device 110 may trigger an invocation to the summary plug-in in response to the presentation condition triggering the health consultation summary 250 being satisfied. In some embodiments, some presentation constraints and presentation policies may also be configured for content specifically related to the fields. For example, the configuration presents relevant content in the form of natural language to improve readability for the user 140. The content corresponding to the finally extracted field cannot exceed the content involved in the interaction information. The highlighted content in the content may be independent of each other. Secondary information may be supplemented based on “medical specific terms” in the content, and the like.

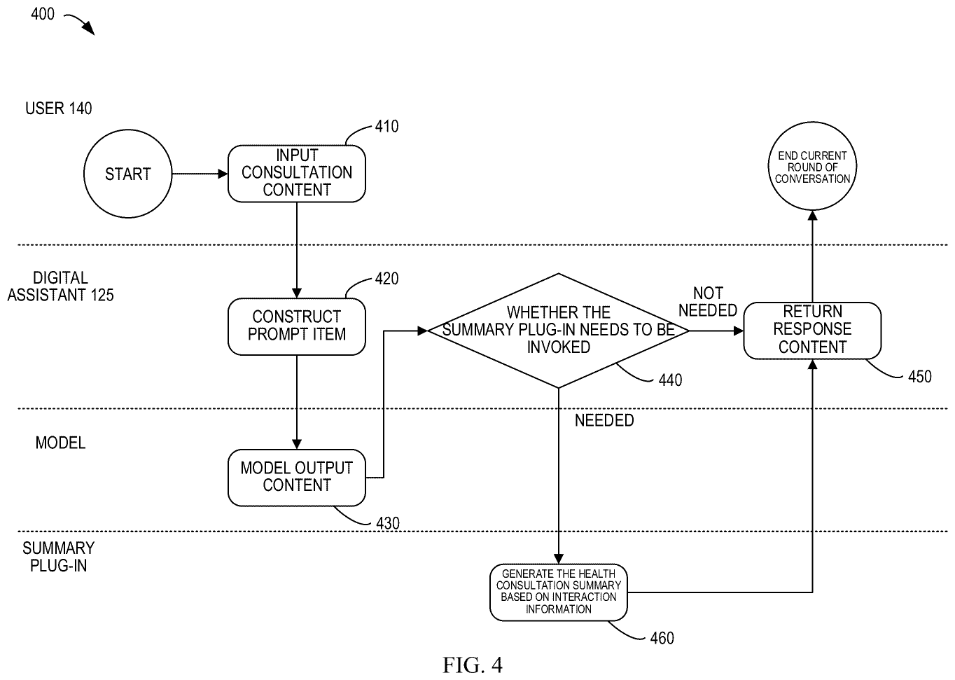

For ease of understanding, examples are given with the digital assistant 125 providing consultation services based on the model. Reference may be made to FIG. 4, which illustrates a schematic diagram of a process 400 of a dialog between a user 140 and a digital assistant 125 according to some embodiments of the present disclosure. For ease of understanding, in this example, an invocation to the summary plug-in may be determined by the digital assistant 125.

At block 410, the user 140 may input the content of the expected consultation to the digital assistant 125.

At block 420, the digital assistant 125 may construct a prompt input based on the consultation content input by the user 140 to use the model based on the prompt input.

At block 430, the model may input the response content to be used for the consultation content based on the prompt input provided by the digital assistant 125.

At block 440, for example, the digital assistant 125 may determine whether the summary plug-in needs to be invoked, for example, judging whether the consultation content, the response content includes the key information, the total number of rounds of the dialog, and the like.

If the determination is not needed, the digital assistant 125 may return the proposed response content as the actually used response content to the user 140 at block 450 to end the dialog of the current round.

If the judgment is needed, the digital assistant 125 may invoke the summary plug-in to generate the health consultation summary 250 with the summary plug-in at block 460.

Further, after generating the health consultation summary 250, the digital assistant 125 may add the health consultation summary 250 to the response content, and at block 450 return the original response content and the health consultation summary 250, or alternatively, use the health consultation summary 250 as an alternative to the proposed response content, to the user 140. The summary plug-in can dynamically determine, according to the information completeness of the current interaction information and the depth of the dialog content, whether the timing to give a conclusion is reached. Thus, the generation of the report is triggered only in a scenario where sufficient necessary information is collected and information of the user 140 is not excessively collected.

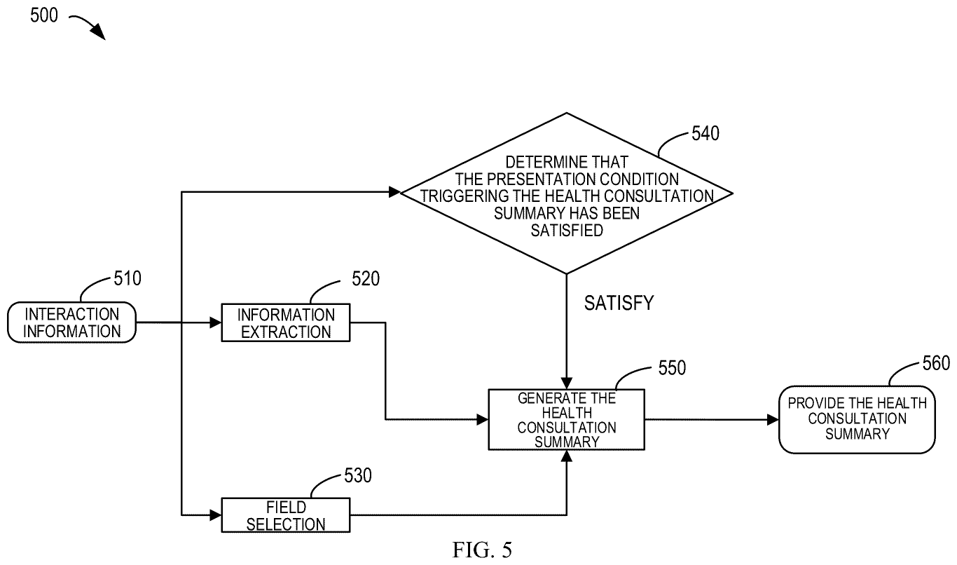

For ease of understanding, the process of judging whether to present the health consultation summary 250, generating the health consultation summary 250, and providing the health consultation summary 250 to the user 140 in the embodiments of the present disclosure may further refer to FIG. 5. FIG. 5 illustrates a schematic diagram of an architecture 500 that provides a health consultation summary according to some embodiments of the present disclosure.

In block 540, the terminal device 110 may determine, based on the content of the interaction information 110, that the presentation condition triggering the health consultation summary has been satisfied. If satisfied, the terminal device 110 may extract, for example, key information in the interaction information 110 in block 520, and perform field selection based on the interaction information in block 530.

Further, at block 550, the terminal device 110 may generate the health consultation summary 250 according to the content extracted in block 520 (e.g., the result obtained by processing the valid information 350 of each sentence in each field through blocks 360 and 370) and the field selected by block 530. Further, at block 560, the terminal device 110 may provide the health consultation summary 250 to the user 140. Therefore, it is ensured that the generated report content is accurate and understandable, and can be better understood by the user 140.

In some embodiments, to facilitate the user 140 to obtain more information, the terminal device 110 may further associate more information in the health consultation summary 250 through a manner of an entry link. For example, the terminal device 110 may annotate at least one entity term in the health consultation summary 250 with a corresponding entry link (for example, for the entry 255-1). The user 140 may jump to the entry page corresponding to the entity term by, for example, clicking the entry 255-1. The user 140 may obtain more relevant information of the entry based on the entry page. Therefore, more information content may be provided for the user 140 by means of an entry link jump.

In some embodiments, the terminal device 110 may also provide an access link of the health consultation summary 250 or an access link to the interaction information associated with the health consultation summary 250 in the health consultation summary 250. For example, in a page associated with the health consultation summary 250, the terminal device 110 may also provide an access link (e.g., access link 256-1) in the display area 256. The user 140 may obtain the health consultation summary 250 or the interaction information associated with the health consultation summary 250 by clicking the access link. Thus, the user 140 may store the health consultation summary 250 or the interaction information associated with the health consultation summary 250 by saving the access link, and conveniently obtain and share the health consultation summary 250 the interaction information associated with the health consultation summary 250 with the access link when there is a need or requirement. In some embodiments, the terminal device 110 may further provide a control 256-2 for generating a two-dimensional code through an independent page, or in the display area 256, and the two-dimensional code generated based on the control 256-2 may be used to obtain the health consultation summary 250 and the interaction information associated with the health consultation summary 250. Therefore, after generating the two-dimensional code, the user 140 may share and obtain the health consultation summary 250 and the interaction information associated with the health consultation summary 250 by “scanning the code” or sharing the two-dimensional code.

In some embodiments, the terminal device 110 may also provide, in the display area 256, an interaction information (e.g., session record) access portal associated with the health consultation summary 250 for access. Therefore, the user 140 or another related parties may quickly obtain the interaction information referenced when generating the health consultation summary 250 through the access portal.

In some embodiments, after a health consultation summary 250 is provided to the user 140. The terminal device 110 may also provide the user 140 with at least one interaction option for the health consultation summary 250. This enables the user 140 to utilize interaction options to perform desired interaction operation on the interaction options. In some embodiments, the at least one interaction operation corresponding to the at least one interaction option includes at least one of the following: a copy operation, an export to a local operation, a share operation, and a print operation. Therefore, the user 140 may complete the operations such as copying, exporting to local, sharing, printing and the like for the health consultation summary 250 through the interaction operation on the interaction options, so as to facilitate the user 140 to actually use the health consultation summary 250 according to the requirement.

Accordingly, the terminal device 110 may perform, in response to receiving the selection of the target interaction option in the at least one interaction option, the target interaction operation corresponding to the target interaction option on the health consultation summary 250, to meet the requirement of the user 140.

For example, refer to FIG. 2E. The user 140 may call a set of interaction options 260 through a preset operation (e.g., a long press, a mouse right-click in an area for presenting health consultation summary 250 in the interface 150, etc.) for the health consultation summary 250. The set of interaction options 260 includes an interaction option 261 for copying the health consultation summary 250, an interaction option 262 for exporting the health consultation summary 250 to the local, an interaction option 263 for sharing the health consultation summary 250, and an interaction option 264 for printing the health consultation summary 250. The user 140 may select at least one option of the set of interaction options 260 based on the requirement to instruct the terminal device 110 to complete the corresponding operation.

In some embodiments, the user 140 may also be provided with the health consultation summary 250 and a “lookback content” of the interaction history associated with the health consultation summary 250. For example, the user 140 may request the lookback of the health consultation summary 250, interaction history associated with the health consultation summary 250, by, for example, sending a lookback request to the digital assistant 125 after completion of the consultation for a period of time. The terminal device 110 may, in response to detecting the viewing request for the health consultation of the user 140, provide the health consultation summary 250; and in response to receiving the viewing request for the interaction history of the health consultation, provide the interaction information between the user 140 and the digital assistant 125 in the health consultation. Therefore, after the consultation is completed, the user 140 may obtain the historical consultation situation by using the “lookback” function, so as to prevent the user 140 from repeatedly consulting for the same question and improve the use experience of the user 140.

In some embodiments, the user 140 may be provided with a health consultation summary 250 and an interaction history associated with the health consultation summary 250 at the same time. The terminal device 110 may choose to preferentially present the health consultation summary 250 (e.g., the initial presentation location of the interface 150, hover at the content associated with the health consultation summary 250) for the user 140 when the user 140 requests a lookback. In this way, after the user 140 may preferentially obtain the concluding health consultation summary 250, the user 140 may browse through the related session process, thereby improving the browsing efficiency of the user 140.

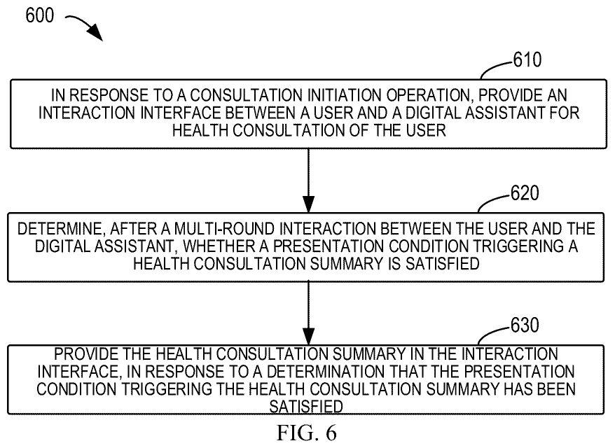

FIG. 6 illustrates a flowchart of a process 600 for information interaction according to some embodiments of the present disclosure. The process 600 may be implemented at the terminal device 110. The process 600 is described below with reference to FIG. 1.

At block 610, in response to a consultation initiation operation, provide an interaction interface between a user and a digital assistant for health consultation of the user.

At block 620, determine, after a multi-round interaction between the user and the digital assistant, whether a presentation condition triggering a health consultation summary is satisfied.

At block 630, provide the health consultation summary in the interaction interface, in response to a determination that the presentation condition triggering the health consultation summary has been satisfied.

In an embodiment of the present disclosure, the health consultation summary has a structured format and is generated based at least on interaction information between the user and the digital assistant.

In some embodiments, determining whether the presentation condition triggering the health consultation summary is satisfied comprises: determining whether the presentation condition triggering the health consultation summary is satisfied based on at least one of: detecting a keyword for triggering the health consultation summary from the interaction information, or determining that the health consultation is able to obtain a predicted consultation conclusion based on the interaction information.

In some embodiments, providing the health consultation summary in the interaction interface comprises: presenting a request for confirmation of an end of the health consultation in the interaction interface, in response to a determination that the presentation condition triggering the health consultation summary has been satisfied; and in response to receiving the confirmation of the end of the health consultation by the user, providing the health consultation summary in the interaction interface.

In some embodiments, the process 600 further comprises: generating the health consultation summary by: selecting at least one field from a plurality of predetermined fields based on the interaction information between the user and the digital assistant, each field indicating a type of information to be filled in; and filling in the selected at least one field based on key information extracted from the interaction information to obtain the health consultation summary.

In some embodiments, selecting the at least one field from the plurality of predetermined fields comprises: determining a disease prediction corresponding to the health consultation of the user based on the interaction information between the user and the digital assistant; and selecting the at least one field from the plurality of predetermined fields based at least on the disease prediction.

In some embodiments, the key information is extracted from at least one of: a textual dialog between the user and the digital assistant, or non-textual information input by the user.

In some embodiments, providing the health consultation summary in the interaction interface comprises: triggering an invocation to a summary plug-in in response to a determination that the presentation condition triggering the health consultation summary has been satisfied, the summary plug-in being configured to generate the health consultation summary based on the interaction information between the user and the digital assistant; and providing the health consultation summary generated by the summary plug-in.

In some embodiments, the process 600 further comprises: in response to detecting an interaction triggering operation on the health consultation summary, providing at least one interaction option for the health consultation summary, each interaction option corresponding to an interaction operation for the health consultation summary; and in response to receiving a selection of a target interaction option in the at least one interaction option, performing a target interaction operation corresponding to the target interaction option on the health consultation summary.

In some embodiments, at least one interaction operation corresponding to the at least one interaction option comprises at least one of: a copy operation, an operation of export to local, a share operation, and a print operation.

In some embodiments, the process 600 further comprises: in response to detecting a viewing request on the health consultation of the user, providing the health consultation summary; and in response to receiving a viewing request on an interaction history of the health consultation, providing the interaction information between the user and the digital assistant in the health consultation.

In some embodiments, at least one entity term is annotated with a respective entry link in the health consultation summary, and each entry link is used to access an entry page corresponding to an entity term.

In some embodiments, at least one of an access link of the health consultation summary, or an access link of the interaction information is provided in a page associated with the health consultation summary.

In some embodiments, the digital assistant generates a response to the user based on a model.

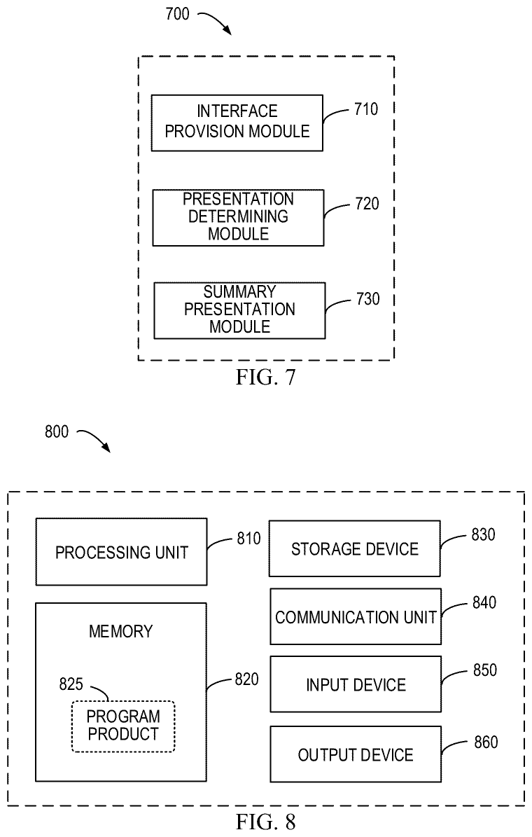

FIG. 7 is a schematic structural block diagram of an apparatus 700 for information interaction according to some embodiments of the present disclosure. The apparatus 700 may be implemented or included in the terminal device 110. The various modules/components in the apparatus 700 may be implemented by hardware, software, firmware, or any combination thereof.

As shown in the figure, the apparatus 700 comprises an interface provision module 710, configured to, in response to a consultation initiation operation, provide an interaction interface between a user and a digital assistant for health consultation of the user; a presentation determining module 720, configured to determine, after a multi-round interaction between the user and the digital assistant, whether a presentation condition triggering a health consultation summary is satisfied; and a summary presentation module 730, configured to provide the health consultation summary in the interaction interface, in response to a determination that the presentation condition triggering the health consultation summary has been satisfied, the health consultation summary having a structured format and being generated based at least on interaction information between the user and the digital assistant.

In some embodiments, determining whether the presentation condition triggering the health consultation summary is satisfied comprises: determining whether the presentation condition triggering the health consultation summary is satisfied based on at least one of: detecting a keyword for triggering the health consultation summary from the interaction information, or determining that the health consultation is able to obtain a predicted consultation conclusion based on the interaction information.

In some embodiments, providing the health consultation summary in the interaction interface comprises: presenting a request for confirmation of an end of the health consultation in the interaction interface, in response to a determination that the presentation condition triggering the health consultation summary has been satisfied; and in response to receiving the confirmation of the end of the health consultation by the user, providing the health consultation summary in the interaction interface.

In some embodiments, the apparatus 700 further comprises: a summary generation module configured to generate the health consultation summary by: selecting at least one field from a plurality of predetermined fields based on the interaction information between the user and the digital assistant, each field indicating a type of information to be filled in; and filling in the selected at least one field based on key information extracted from the interaction information to obtain the health consultation summary.

In some embodiments, selecting the at least one field from the plurality of predetermined fields comprises: determining a disease prediction corresponding to the health consultation of the user based on the interaction information between the user and the digital assistant; and selecting the at least one field from the plurality of predetermined fields based at least on the disease prediction.

In some embodiments, the key information is extracted from at least one of: a textual dialog between the user and the digital assistant, or non-textual information input by the user.

In some embodiments, providing the health consultation summary in the interaction interface comprises: triggering an invocation to a summary plug-in in response to a determination that the presentation condition triggering the health consultation summary has been satisfied, the summary plug-in being configured to generate the health consultation summary based on the interaction information between the user and the digital assistant; and providing the health consultation summary generated by the summary plug-in.

In some embodiments, the apparatus 700 further comprises: an interaction execution module configured to, in response to detecting an interaction triggering operation on the health consultation summary, provide at least one interaction option for the health consultation summary, each interaction option corresponding to an interaction operation for the health consultation summary; and in response to receiving a selection of a target interaction option in the at least one interaction option, perform a target interaction operation corresponding to the target interaction option on the health consultation summary.

In some embodiments, at least one interaction operation corresponding to the at least one interaction option comprises at least one of: a copy operation, an operation of export to local, a share operation, and a print operation.

In some embodiments, the apparatus 700 further comprises: an information provision module configured to, in response to detecting a viewing request on the health consultation of the user, provide the health consultation summary; and in response to receiving a viewing request on an interaction history of the health consultation, provide the interaction information between the user and the digital assistant in the health consultation.

In some embodiments, at least one entity term is annotated with a respective entry link in the health consultation summary, and each entry link is used to access an entry page corresponding to an entity term.

In some embodiments, at least one of an access link of the health consultation summary, or an access link of the interaction information is provided in a page associated with the health consultation summary.

In some embodiments, the digital assistant generates a response to the user based on a model.

FIG. 8 illustrates a block diagram of an electronic device 800 in which one or more embodiments of the present disclosure may be implemented. It should be understood that the electronic device 800 illustrated in FIG. 8 is merely illustrative and should not constitute any limitation on the functionality and scope of the embodiments described herein. The electronic device 800 shown in FIG. 8 may be configured to implement the terminal device 110 in FIG. 1.

As shown in FIG. 8, the electronic device 800 is in the form of a general-purpose electronic device. Components of the electronic device 800 may include, but are not limited to, one or more processors or processing units 810, a memory 820, a storage device 830, one or more communication units 840, one or more input devices 850, and one or more output devices 860. The processing unit 810 may be an actual or virtual processor and capable of performing various processes according to programs stored in the memory 820. In multiprocessor system, multiple processing units execute computer-executable instructions in parallel to improve parallel processing capabilities of electronic device 800.

The electronic device 800 typically includes a plurality of computer storage media. Such media may be any available media accessible to the electronic device 800, including, but not limited to, volatile and non-volatile media, removable and non-removable media. The memory 820 may be volatile memory (e.g., registers, caches, random access memory (RAM)), non-volatile memory (e.g., read-only memory (ROM), electrically erasable programmable read-only memory (EEPROM), flash memory), or some combination thereof. Storage device 830 may be a removable or non-removable medium and may include a machine-readable medium, such as a flash drive, magnetic disk, or any other medium, which may be capable of storing information and/or data and may be accessed within electronic device 800.

The electronic device 800 may further include additional removable/non-removable, volatile/non-volatile storage media. Although not shown in FIG. 8, a disk drive for reading or writing from a removable, nonvolatile magnetic disk (e.g., a “floppy disk”) and an optical disk drive for reading or writing from a removable, nonvolatile optical disk may be provided. In these cases, each drive may be connected to a bus (not shown) by one or more data media interface. The memory 820 may include a computer program product 825 having one or more program modules configured to perform various methods or actions of various embodiments of the present disclosure.

The communication unit 840 is configured to communicate with another electronic device through a communication medium. Additionally, the functionality of components of the electronic device 800 may be implemented in a single computing cluster or multiple computing machines capable of communicating over a communication connection. Thus, the electronic device 800 may operate in a networked environment using logical connections with one or more other servers, network personal computers (PCs), or another network node.

The input device 850 may be one or more input devices, such as a mouse, a keyboard, a trackball, or the like. The output device 860 may be one or more output devices, such as a display, a speaker, a printer, or the like. The electronic device 800 may also communicate with one or more external devices (not shown) through the communication unit 840 as needed, external devices such as storage devices, display devices, etc., communicate with one or more devices that enable a user to interact with the electronic device 800, or communicate with any device (e.g., a network card, a modem, etc.) that enables the electronic device 800 to communicate with one or more other electronic devices. Such communication may be performed via an input/output (I/O) interface (not shown).

According to example implementations of the present disclosure, there is provided a computer-readable storage medium having computer-executable instructions stored thereon, wherein the computer-executable instructions are executed by a processor to implement the method described above. According to example implementations of the present disclosure, a computer program product is further provided, the computer program product being tangibly stored on a non-transitory computer-readable medium and including computer-executable instructions, the computer-executable instructions being executed by a processor to implement the method described above.

Aspects of the present disclosure are described herein with reference to flowcharts and/or block diagrams of methods, apparatuses, devices, and computer program products implemented in accordance with the present disclosure. It should be understood that each block of the flowchart and/or block diagram, and combinations of blocks in the flowcharts and/or block diagrams, may be implemented by computer readable program instructions.

These computer-readable program instructions may be provided to a processing unit of a general purpose computer, special purpose computer, or other programmable data processing apparatus to produce a machine, such that the instructions, when executed by a processing unit of a computer or other programmable data processing apparatus, produce means to implement the functions/actions specified in the flowchart and/or block diagram. These computer-readable program instructions may also be stored in a computer-readable storage medium that cause the computer, programmable data processing apparatus, and/or other devices to function in a particular manner, such that the computer-readable medium storing instructions includes an article of manufacture including instructions to implement aspects of the functions/actions specified in the flowchart and/or block diagram(s).

The computer-readable program instructions may be loaded onto a computer, other programmable data processing apparatus, or other apparatus, such that a series of operational steps are performed on a computer, other programmable data processing apparatus, or other apparatus to produce a computer-implemented process such that the instructions executed on a computer, other programmable data processing apparatus, or other apparatus implement the functions/actions specified in the flowchart and/or block diagram block or blocks.

The flowchart and block diagrams in the figures show architecture, functionality, and operation of possible implementations of systems, methods, and computer program products according to various implementations of the present disclosure. In this regard, each block in the flowchart or block diagram may represent a module, program segment, or portion of an instruction that includes one or more executable instructions for implementing the specified logical function. In some alternative implementations, the functions noted in the blocks may also occur in a different order than noted in the figures. For example, two consecutive blocks may actually be performed substantially in parallel, which may sometimes be performed in the reverse order, depending on the functionality involved. It is also noted that each block in the block diagrams and/or flowchart, as well as combinations of blocks in the block diagrams and/or flowchart, may be implemented with a dedicated hardware-based system that performs the specified functions or actions, or may be implemented in a combination of dedicated hardware and computer instructions.

Various implementations of the present disclosure have been described above, which are illustrative, not exhaustive, and are not limited to the implementations disclosed. Many modifications and variations will be apparent to those of ordinary skill in the art without departing from the scope and spirit of the various implementations illustrated. The selection of the terms used herein is intended to best explain the principles of the implementations, practical applications, or improvements to techniques in the marketplace, or to enable others of ordinary skill in the art to understand the various implementations disclosed herein.

I/We claim:

1. A method of information interaction, comprising:
in response to a consultation initiation operation, providing an interaction interface between a user and a digital assistant for health consultation of the user;
determining, after a multi-round interaction between the user and the digital assistant, whether a presentation condition triggering a health consultation summary is satisfied; and
providing the health consultation summary in the interaction interface, in response to a determination that the presentation condition triggering the health consultation summary has been satisfied, the health consultation summary having a structured format and generated based at least on interaction information between the user and the digital assistant.

2. The method of claim 1, wherein determining whether the presentation condition triggering the health consultation summary is satisfied comprises:
determining whether the presentation condition triggering the health consultation summary is satisfied based on at least one of:
detecting a keyword for triggering the health consultation summary from the interaction information, or
determining that the health consultation is able to obtain a predicted consultation conclusion based on the interaction information.

3. The method of claim 1, wherein providing the health consultation summary in the interaction interface comprises:
presenting a request for confirmation of an end of the health consultation in the interaction interface, in response to a determination that the presentation condition triggering the health consultation summary has been satisfied;
in response to receiving the confirmation of the end of the health consultation by the user, providing the health consultation summary in the interaction interface.

4. The method of claim 1, further comprising: generating the health consultation summary by:
selecting at least one field from a plurality of predetermined fields based on the interaction information between the user and the digital assistant, each field indicating a type of information to be filled in; and
filling in the selected at least one field based on key information extracted from the interaction information to obtain the health consultation summary.

5. The method of claim 4, wherein selecting the at least one field from the plurality of predetermined fields comprises:
determining a disease prediction corresponding to the health consultation of the user based on the interaction information between the user and the digital assistant; and
selecting the at least one field from the plurality of predetermined fields based at least on the disease prediction.

6. The method of claim 4, wherein the key information is extracted from at least one of: a textual dialog between the user and the digital assistant, or non-textual information input by the user.

7. The method of claim 1, wherein providing the health consultation summary in the interaction interface comprises:
triggering an invocation to a summary plug-in in response to a determination that the presentation condition triggering the health consultation summary has been satisfied, the summary plug-in being configured to generate the health consultation summary based on the interaction information between the user and the digital assistant; and
providing the health consultation summary generated by the summary plug-in.

8. The method of claim 1, further comprising:
in response to detecting an interaction triggering operation on the health consultation summary, providing at least one interaction option for the health consultation summary, each interaction option corresponding to an interaction operation for the health consultation summary; and
in response to receiving a selection of a target interaction option in the at least one interaction option, performing a target interaction operation corresponding to the target interaction option on the health consultation summary.

9. The method of claim 8, wherein at least one interaction operation corresponding to the at least one interaction option comprises at least one of: a copy operation, an operation of export to local, a share operation, and a print operation.

10. The method of claim 1, further comprising:
in response to detecting a viewing request on the health consultation of the user, providing the health consultation summary; and
in response to receiving a viewing request on an interaction history of the health consultation, providing the interaction information between the user and the digital assistant in the health consultation.

11. The method of claim 1, wherein at least one entity term is annotated with a respective entry link in the health consultation summary, and each entry link is used to access an entry page corresponding to an entity term.

12. The method of claim 1, wherein at least one of an access link of the health consultation summary, or an access link of the interaction information is provided in a page associated with the health consultation summary.

13. The method of claim 1, wherein the digital assistant generates a response to the user based on a model.

14. An electronic device comprises:
at least one processing unit; and
at least one memory coupled to the at least one processing unit and storing instructions for execution by the at least one processing unit, the instructions, when executed by the at least one processing unit, causing the electronic device to perform a method comprising:
in response to a consultation initiation operation, providing an interaction interface between a user and a digital assistant for health consultation of the user;
determining, after a multi-round interaction between the user and the digital assistant, whether a presentation condition triggering a health consultation summary is satisfied; and
providing the health consultation summary in the interaction interface, in response to a determination that the presentation condition triggering the health consultation summary has been satisfied, the health consultation summary having a structured format and generated based at least on interaction information between the user and the digital assistant.

15. The electronic device of claim 14, wherein determining whether the presentation condition triggering the health consultation summary is satisfied comprises:
determining whether the presentation condition triggering the health consultation summary is satisfied based on at least one of:
detecting a keyword for triggering the health consultation summary from the interaction information, or
determining that the health consultation is able to obtain a predicted consultation conclusion based on the interaction information.

16. The electronic device of claim 14, wherein providing the health consultation summary in the interaction interface comprises:
presenting a request for confirmation of an end of the health consultation in the interaction interface, in response to a determination that the presentation condition triggering the health consultation summary has been satisfied;
in response to receiving the confirmation of the end of the health consultation by the user, providing the health consultation summary in the interaction interface.

17. The electronic device of claim 14, wherein the method further comprises: generating the health consultation summary by:
selecting at least one field from a plurality of predetermined fields based on the interaction information between the user and the digital assistant, each field indicating a type of information to be filled in; and
filling in the selected at least one field based on key information extracted from the interaction information to obtain the health consultation summary.

18. The electronic device of claim 17, wherein selecting the at least one field from the plurality of predetermined fields comprises:
determining a disease prediction corresponding to the health consultation of the user based on the interaction information between the user and the digital assistant; and
selecting the at least one field from the plurality of predetermined fields based at least on the disease prediction.

19. The electronic device of claim 17, wherein the key information is extracted from at least one of: a textual dialog between the user and the digital assistant, or non-textual information input by the user.

20. A computer-readable storage medium having a computer program stored thereon, the computer program being executable by a processor to implement a method comprising:
in response to a consultation initiation operation, providing an interaction interface between a user and a digital assistant for health consultation of the user;
determining, after a multi-round interaction between the user and the digital assistant, whether a presentation condition triggering a health consultation summary is satisfied; and
providing the health consultation summary in the interaction interface, in response to a determination that the presentation condition triggering the health consultation summary has been satisfied, the health consultation summary having a structured format and generated based at least on interaction information between the user and the digital assistant.
